(19) United States
(12) Patent Application Publication
(10) Pub. No.: US 2022/0244267 A1
(43) Pub. Date: Aug. 4, 2022

(54) METHOD OF PREPARING A SAMPLE FOR A DIAGNOSTIC ASSAY

(71) Applicant: PROLIGHT DIAGNOSTICS AB, Lund (SE)

(72) Inventors: Ben Metcalf, Melbourn, Royston Hertfordshire (GB); Steven Wakefield, Melbourn, Royston Hertfordshire (GB); Hans Hoppe, Melbourn, Royston Hertfordshire (GB); Wenshu Xu, Melbourn, Royston Hertfordshire (GB); Keiko Yata, Melbourn, Royston Hertfordshire (GB); Cara Deal, Melbourn, Royston Hertfordshire (GB)

(73) Assignee: PROLIGHT DIAGNOSTICS AB, Lund (SE)

(21) Appl. No.: 17/610,891

(22) PCT Filed: May 12, 2020
(86) PCT No.: PCT/GB2020/051158

§ 371 Date: Nov. 12, 2021

(30) Foreign Application Priority Data
May 13, 2019 (GB) 1906722.2

(51) Int. Cl. G01N 33/68; G01N 1/40

(52) U.S. Cl. CPC G01N 33/68; G01N 1/405

(57) ABSTRACT

The present invention relates to a method of preparing a sample for subsequent use in a diagnostic or analytical assay by concentrating the target analyte and/or removing interfering factors from the sample matrix. The present invention also relates to a fluidic device comprising a capture and release system connected to an immunodetection system.

### FIELD OF INVENTION

The present invention relates to a method of preparing a sample for subsequent use in a diagnostic or analytical assay by concentrating the target analyte and/or removing interfering factors from the sample matrix.

### BACKGROUND

One solution to the problem of the long turnaround that many diagnostic tests take is the development of point-of-care (PoC) testing. PoC enables faster access to diagnostic test results, allowing for more rapid clinical decision making. PoC systems reduce the complex, labour-intensive, skilled diagnostic workflows traditionally carried out in central laboratories into a simple, automated reusable test instrument that can perform sample-to-answer diagnostic tests using a low-cost, self-contained, disposable testing device, e.g. a cartridge or strip containing analytical elements.

Diagnostic assays are often first developed in a micro-titre plate format to allow for high-throughput experimental testing of various conditions. Developing an assay to perform well with clinical biological samples from multiple patients' presents a particular challenge because such samples can include multiple inhibitory or interfering substances which can further be different between different patient groups. This naturally leads to a substantially increased development effort as the assay must be developed against multiple different sample types to work simultaneously.

A key challenge in developing diagnostics assays for use in PoC systems is translating the initial assay development work undertaken into a format suitable for integration into a disposable test device. The limitations of such disposable test devices (for example, restrictions on the fluid manipulation steps available) often lead to systems that require significant re-development of the assay to fit within whatever test format is desired. This is often accompanied by a subsequent reduction in achievable sensitivity.

This leads to a second key challenge—achieving the required sensitivity of the diagnostic test on the disposable fluidic device. Previous systems have overcome this by trying to amplify the signal at the end, either through re-engineering of the bio-chemistry or by using additional detection mechanisms such as surface plasmon resonance (SPR) and electro-chemical detection. This can often result in deviation from previously developed assays which then require significant further development.

A third key challenge is the diagnostic test variability introduced by differences between individual biological samples. This often requires significant development to improve this response, often requiring development to be conducted on individual patient samples which can significantly increase both the development time and cost of the assay.

Current solutions include the development of a new diagnostic assay around the target format for the disposable test device. Examples of this approach include Abbott iStat, Philips Minicare, Siemens Stratus and Alere Triage. However, the development of such devices comes at a significant development cost and increases the associated development time due to the reduced experimental throughput possible by developing assays on a single-use platform. Alternatively, disposable test formats may be developed that enable the translation of an existing diagnostic assay. Examples of this approach include LSI Medience Pathfast, Biomerieux Vidas and Radiometer AQT90 FLEX. However, this approach comes with the risk that the sensitivity of the assay is compromised resulting in an inaccurate reading of the target analyte.

Therefore, there is a particular need in the art for a PoC-compatible system that can perform a highly-sensitive diagnostic test without requiring significant re-development of an existing diagnostic assay.

### SUMMARY OF THE INVENTION

In an aspect, the present invention provides a method of preparing a sample for carrying out a diagnostic or analytical assay, wherein a sample to be assayed is first bought into contact with a housing unit that comprises an ionically charged surface; wherein a target analyte is retained on said membrane and the other constituents of the sample pass through said surface freely; releasing the target analyte from the surface by introduction of an elution fluid, which is collected via an outlet collection port for a diagnostic or analytical assay to be performed.

In a second aspect, the present invention provides a fluidic device comprising a capture and release system connected to an immunodetection system via fluidic channels to enable the aforementioned method of preparing a sample for carrying out a diagnostic or analytical assay.

It will be appreciated that the present invention not only provides for a method of preparing a sample for improved accuracy and sensitivity in a diagnostic/analytical assay, but also provides for a device capable of performing both the processing and the subsequent analytical assay in a format suitable for PoC testing.

### DESCRIPTION OF FIGURES

The invention will now be described with reference to the accompanying figures, in which:

FIG. 1 shows a fluidic device comprising both the capture and release system and immunodetection system connected to each other via fluidic channels;

FIG. 2 shows the basic principle of the capture and release system involving the use of an ionically charged surface; the adjustment of the target analyte charge so that the charge is sufficiently different from other analytes within the sample in order for the target analyte to be captured on the ionically charged surface; an optional wash step in where analytes not of interest are further removed from the ionically charged surface due to their similar ionic charge to the ionically charged surface; and the subsequent elution of the target analyte into an elution fluid;

FIG. 3 shows the use of an ionically charged surface to capture target analytes which present an interacting surface with predominantly opposite charged regions from within a fluid that is flowed through the membrane;

FIG. 4 shows the process of capturing and releasing a target analyte, e.g. the cardiac protein troponin, from a human serum or plasma sample via adjusting the ionic conditions of the sample, contacting the sample with an ionically charged surface and subsequent release of the target analyte via further alteration of the ionic conditions of a sample with an elution fluid;

FIG. 5 shows an external design of a fluidic housing unit which supports the capture and release ionically charged surface such that it can be integrated within a disposable test device;

FIG. 6 shows an internal design of a fluidic housing unit which supports the capture and release ionically charged surface such that it can be integrated within a disposable test device;

FIG. 7 shows a fluidic device with various fluidic channels, actuating valves, input and output ports to direct fluid toward and away from the ionically charged surface;

FIG. 8 shows a design of elastomeric membranes to create fluidic channels which can be operational under both positive and negative pressure;

FIG. 9 shows a device designed for use as an immunodetection system;

FIG. 10 shows the central reaction chamber within the immunodetection system including a possible arrangement of magnets;

FIG. 11 shows the positioning of magnets and alteration of their activation states within the central reaction chamber to collect the magnetic particles in one location within the immunodetection system;

FIG. 12 shows the positioning of magnets within the central reaction chamber to improve the mixing of reactants within the immunodetection system;

FIG. 13 shows the addition of second and third chambers to the first central reaction chamber within the immunodetection system;

FIG. 14 shows the process by which fluid may be transferred between different reaction chambers within the immunodetection system;

FIG. 15 shows an analytical device wherein the capture and release system and the immunodetection system are located on the same device.

### DETAILED DESCRIPTION

The present invention aims to improve test sensitivity by pre-processing of a sample, opposed to amplifying any test output signal at the end of the test. Advantages of pre-processing a sample are two-fold; firstly, it provides an opportunity for the removal of the interfering factors from the sample and secondly, it allows concentration of the analyte such that the input to the diagnostic assay now has fewer interfering substances and is at a detectable concentration.

Such an approach allows the pre-processed sample (that is, one that increases the effective analyte concentration in the sample whilst reducing the level if interfering substances present within it) to be used as the input to an existing diagnostic assay that has been translated onto a disposable fluidic device and retain or improve the performance of this assay as achieved in a micro-titre plate format.

The sample pre-processing reduces the performance requirements of the diagnostic assay running on the disposable test device and so allows a wider range of more simple devices and test methods to be used, for example by reducing the need for complex and costly detection schemes and instead relying on simple luminescence measurements. By reducing the necessary complexity of the ‘back-end’ test device, the manufactured cost of this can be reduced and, in turn, reduces the development required to translate additional assays on to this device.

A further benefit is that by removing some of the interfering substances before the sample is added to the assay; the robustness of the assay is improved and as a result reduces the variability of the assay between different patients, thereby reducing the amount of extra assay development work that would normally be necessary to achieve this.

In one aspect, the present invention provides a method of preparing a sample for carrying out a diagnostic or analytical assay, wherein a sample to be assayed is first brought into contact with a housing unit that comprises an ionically charged surface; wherein a target analyte is retained on said surface and the other constituents of the sample pass through said surface freely; releasing the target analyte from the surface by introduction of an elution fluid, which is collected via an outlet collection port for a diagnostic or analytical assay to be performed.

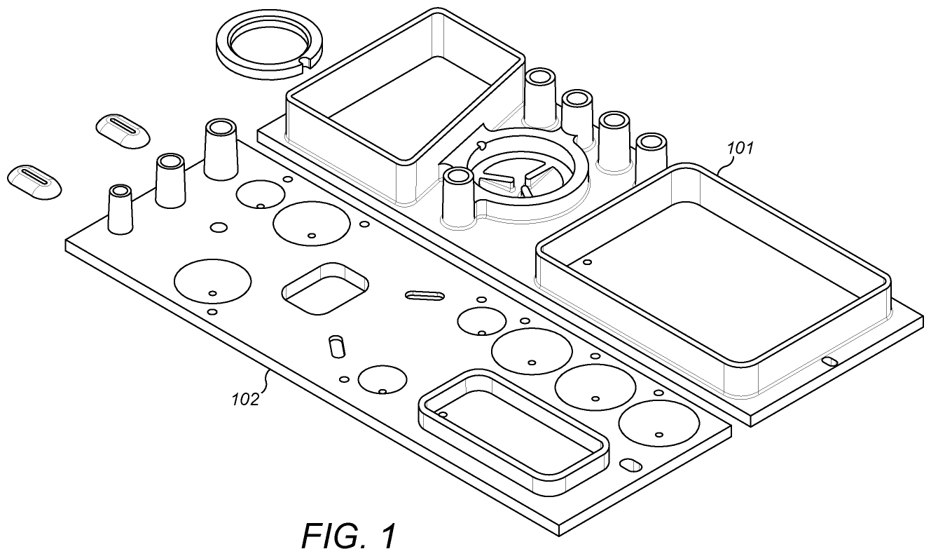

FIG. 1 shows a possible device comprising both the housing unit 101 for containing the ionically charged surface and a connected analytical assay, specifically an immunodetection system 102.

For clarity, the term ‘ionic charge’ refers to the electrical fee of an ion, created by either the gain (negative charge) or loss (positive charge) of one or more electrons from an atom or group of atoms. Ions with a negative charge are termed anions, whereas those with a positive charge are termed cations.

The ionically charged surface used in the present invention can be permeable or semi-permeable to allow the passage of fluid through said surface. The ionically charged surface may have either a permanent or non-permanent acquired ionic charge to achieve the desired effect.

The ionically charged surface may be an ion-exchange membrane or ion-exchange beads. The term ‘ion-exchange membrane’ refers to a semi-permeable membrane that allows the movement of certain ions, whilst restricting the movement of others. An example of such a membrane that would be suitable for use in the present invention is the Mustang Q membrane from Pall Corporation. Alternatively, ion-exchange beads may be used to achieve the same purpose. One example of ion-exchange magnetic beads suitable for this purpose are silica-coated magnetic beads which are modified to carry diethylaminoethyl (DEAF) moieties on their surface (Cat. No. VVHM-CO15, Creative Diagnostics). Molecules with surfaces of opposite charge will be attracted by the bead surface and this interaction can be used to enrich such molecules through magnetic separation followed by a wash and elution step, for example, using high salt (2 M NaCl) solutions to elute enriched molecules off the bead surface. Another product which could be used for the same purpose are Amsbio's MagSi-WCX which carry a weak cation exchange surface.

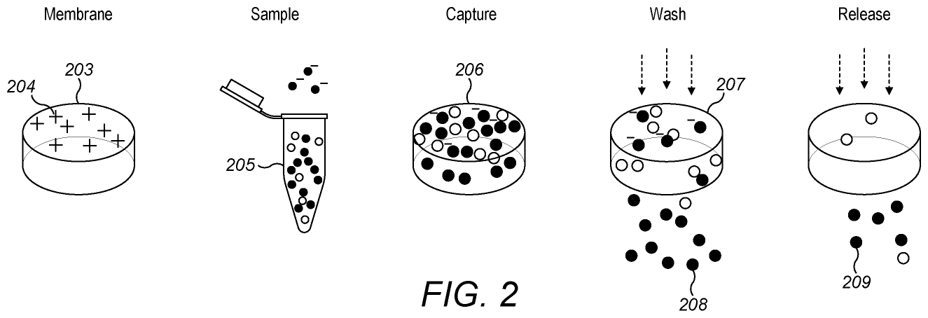

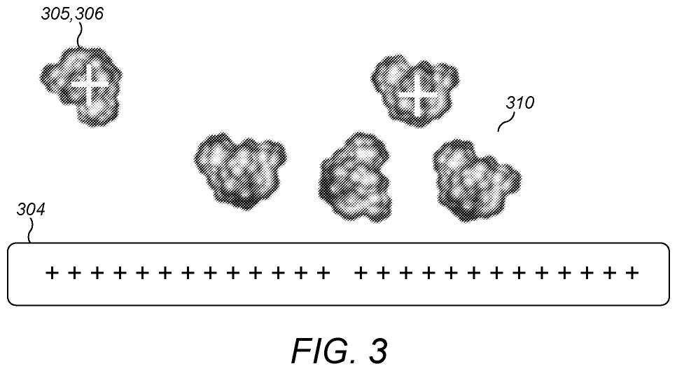

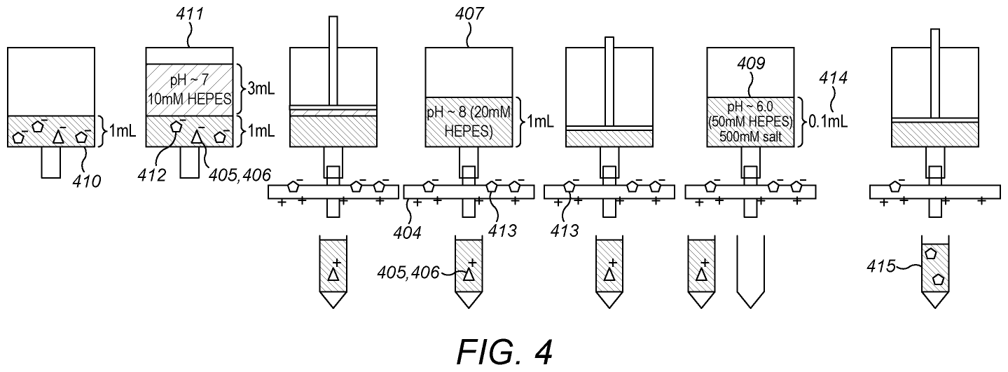

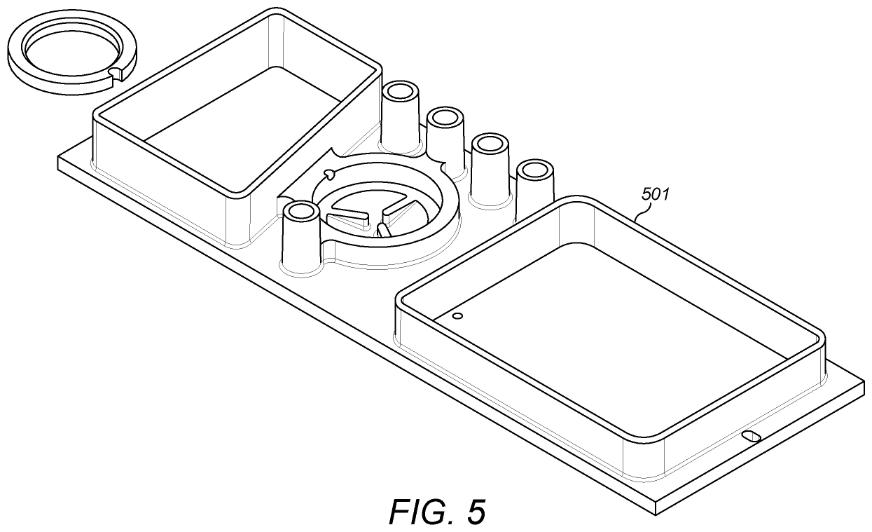

FIGS. 2, 3 and 4 show how an ionically charged membrane may be utilised in the presently claimed method. The ion-exchange membrane 203 (or the ion-exchange beads) may carry a permanently acquired positive or negative ionic charge 204. In this way, the ionically charged surface is able to retain or ‘capture’ substances within a sample which may have an opposite charge, or predominantly opposite charge, to said surface on one of their exposed sides 205, 206. The charge of the ion-exchange surface will be understood to depend on the charge of the target analyte in question, for example, if the target analyte has a predominantly negative charge exposed on at least one surface, the ionically charged membrane will be engineered to carry a positive charge. Examples of suitable surface chemistries include ones modified for strong (quaternary ammonium) and weak (diethylaminoethyl) anion exchange and strong (sulfonic acid) or weak (carboxymethyl) cation exchange. The ionically charged surface may be manufactured with a positive or negative charge. Alternatively, the ionic charge of the surface may be altered, for example, by using an electric current.

Similar to the alteration of the charge of the ‘capture’ surface, the sample itself may be adjusted to carry either a negative or positive charge. As above, the charge imparted to the sample will depend on the charge of the ‘capture’ surface in question. For the sample to acquire a different charge, the sample may be diluted with a buffer which may have a number of parameters may altered; these include both the pH and the salt concentration of the sample. The salt concentration is determined by the addition of sodium chloride, however it is understood that other salts may achieve the same effect. Examples of additional salts that could be used in the present invention include; magnesium chloride, potassium chloride, amino acid salts (e.g. glycine chloride), organic acid salts (e.g. sodium citrate) or salts which relate to gases, such as ammonia or CO₂ (HCO₃). Additionally, a buffering agent may be included for the purpose of maintaining the desired pH. Preferably, this buffering agent is HEPES or a phosphate-based agent; however other buffering agents may be used to achieve the same purpose. Additional buffering agents include citrate and MES. It is understood that the molarity of the buffering component may vary as long as it holds the desired pH of the sample.

Assigning the target analyte 310 a particular charge allows the target analyte to have a sufficiently different charge from a variety of other analytes within the sample 305, 306, many of which are of no interest in the diagnostic process and merely act as contaminating substances. Accordingly, whilst the target of interest is retained on the ‘capture’ surface, other analytes which are of no interest may pass through the surface freely 208 and are accordingly disposed of to avoid any contamination of the ‘purified’ sample containing the isolated target analyte 310.

The sample buffer may comprise 10 mM buffering agent, has a pH of 7-9 and/or salt concentration of 0-50 mM, preferably wherein the buffering agent is HEPES 411 or a phosphate-based agent. This particular sample buffer would infer a negative charge 412 upon the target analyte in question if the target analyte contains more chemical groups on at least one surface which de-protonate at this pH than would accept protons and therefore be directed to a positively charged surface wherein it is ‘captured’ on said surface 413. In one example, the analyte could be a protein which contains significantly more glutamate and aspartate residues than lysine, arginine or histidine ones.

In another example, the sample buffer may comprise 10 mM buffering agent, has a pH of 4-6 and/or salt concentration of 0-50 mM, preferably wherein the buffering agent is MES, citrate or a phosphate-based agent. This particular composition infers a positive charge upon the target analyte in question (which has more chemical groups on at least one surface which accept protons than ones which de-protonate at this pH) and therefore is directed to a negatively charged surface wherein it is captured on said surface.

Many analytes, in particular proteins, are known to display a range of surface charges at any given pH due to the equilibrium-based protonation and de-protonation reactions underpinning the charge acquisition effect caused by pH manipulation. As a consequence, the ionically charged surface may retain at least 70% of the target analyte within a sample. The ionically charged surface may further retain at least 75%, at least 80%, at least 85%, at least 90%, at least 91%, at least 92%, at least 93%, at least 94%, at least 95%, at least 96%, at least 97%, at least 98%, at least 99% or 100% of the target analyte. The other constituents of the sample, with the same charge as the ionically charged surface, pass through the surface freely 208.

As the interaction between charged surface and target analyte is ionic, i.e. positively charge groups interacting with negatively charged moieties, other ions in the sample solution (or wash and elution solutions) can compete effectively with the targeted binding of the analyte to the membrane. Consequently, for adsorption at the desired pH to occur, concentrations of other ions should be kept sufficiently low for the majority of binding reactions to occur. For wash solutions, the ionic concentration can typically be raised a little as target analytes once bound by the charged surface can withstand slightly increase salt concentrations for short periods. The principle of ionic interference can then be used deliberately in the elution step where very high salt concentrations, e.g. 1 M NaCl can be used to recover the analyte, even without alteration to the pH of the solution. Change in pH to change the charge of the analyte would however work synergistically with high salt to elute the analyte and in practice, both methods are used simultaneously.

Typically, low-salt solutions suitable for binding target protein analytes exhibit electrical conductivity of 100 μS/cm at 25° C., much higher salt concentrations used for elution can exhibit electrical conductivities of 1,000 μS/cm at 25° C. or higher.

The present invention may also include a wash step 207, 407 to aid the removal of other proteins from the ionically charged surface, further ‘purifying’ the sample containing the target analyte. An example of an appropriate wash solution may be deionised water, PBS, HBS, TBS, all with or without additional salts or surfactants such as Triton, and may include 20 mM HEPES at a pH of 8. As previously described, additional buffering agents may be used if deemed appropriate.

Following the ‘capturing’ of the target analyte onto the ionically charged surface, a second volume of fluid (referred to as the elution fluid 409) may be applied to the ionically charged surface to aid in the release of the target analyte into the elution fluid for the purpose of inputting the sample into a diagnostic or analytical assay. For this ‘release’ process to occur the elution fluid composition is such that the ionic interactions between the target analyte and the charged surface are sufficiently altered, allowing for the release of the previously ‘captured’ analyte into the second fluid.

The elution fluid composition may comprise 20-50 mM buffering agent, has a pH of 4-8 and a salt concentration of 250 mM-1 M, with or without additional surfactants, such as Tween or Triton, preferably wherein the buffering agent is HEPES. As previously highlighted and discussed, the salt may be any salt appropriate for this purpose, for example, sodium chloride, and the buffering agent may instead be phosphate, citrate and/or MES. The range of pH and salt concentration for the elution fluid are independent of each other, but can work synergistically to interrupt the interactions between the analyte and the charged membrane. To recover a negatively charged analyte, or an analyte with at least one negatively charged surface, from a positively charged surface, a low pH (4) can be used with low salt concentration (250 mM), and a high pH (8) should be used with high salt concentration (more than 500 mM). Whereas, to elute a positively charged analyte, or an analyte with at least one positively charged surface, from a negatively charged surface, a low pH (4) should be used with high salt concentration (more than 500 mM), and a high pH (8) can be used with a lower salt concentration (250 mM).

The elution fluid volume may be smaller than that of the sample volume 414. The optimum elution volume is dependent on the surface area of the charged surface and of the void volume in the device. For example, the volume of the elution fluid 409 may be 10× smaller than that of the input sample volume. For instance, if the input sample volume was 1 mL, the elution fluid 409 could be a volume of 100 μL. The elution fluid 409 additionally does not contain many other proteins and therefore has a reduced level of interfering substances present. Therefore, this process effectively increases the concentration of the target analyte 415 by 10X as well as isolating the target analyte of interest.

Alternatively, the elution fluid 409 may be of an equal volume to that of the sample volume, functioning purely as a means to release the target analyte into a fresh fluid with fewer interfering substances present. In this particular embodiment the concentration of the target analyte 310 would not be increased for use in a diagnostic/analytical assay.

The elution fluid 409 containing the target analyte 310 may be collected and bought into contact with the ionically charged surface multiple times. It is envisaged that this process will increase further the amount of target analyte released into the elution fluid. The same surface, for example an ionically charged membrane, can be effectively regenerated after the elution step by applying the 10 mM buffering agent.

The samples for use in the present invention may be obtained either from a human or animal source. Examples of an animal source may include porcine, feline, canine, bovine, equine or murine sources. Therefore, the present invention is envisaged to be of use as a diagnostic/analytical assay in the veterinary field also.

The sample may be a blood sample (plasma or serum), urine sample, sweat sample, tear sample, sputum sample, faecal sample, liquid biopsy sample or spinal fluid sample. The sample may be fresh or frozen (provided the analyte is stable after the freeze-thaw cycle). Different biological samples or frozen samples may need to be pre-filtered through a membrane filter (pore size <0.8 μm) to remove large particles to prevent blockage and encourage ease of movement through the ionically charged surface. It is understood that different biological samples may need diluting to different extents prior to applying to the ionically charged surface due to differing viscosities. Urine, tear and faecal samples may have significantly elevated salt concentrations compared to blood and therefore should be diluted appropriately. It is understood that pH adjustments may also be required to mitigate variations in input samples.

The target analyte or substance may be a protein, a RNA molecule or DNA molecule. The target analyte might also consist of a virus (enveloped, non-enveloped, RNA or DNA virus, or empty viral shells) or a bacterium, or parts thereof, as charges on molecules decorating the external surfaces of viruses or bacteria can interact with charged surfaces in the same way as isolated proteins can do.

The target analyte may be a cardiac protein, preferably wherein the target analyte is troponin, particularly wherein the target analyte is the troponin IC and troponin ICT complexes. Troponin is a known indicator of damage to the heart muscle (the myocardium) and can be measured in the blood to determine if the patient is suffering from a range of cardiovascular issues, examples of these problems may include unstable angina, tachycardia, heart failure and myocardial infarction. Therefore, the inventors of the present application particularly envisage that the currently disclosed method could be used to aid in the diagnosis of cardiovascular disease in humans and/or animals. Additional disease areas which may also benefit are those where a protein biomarker is known to be present at such low concentration in biological fluids that a pre-concentration step would be of benefit in its detection. Preferably, the target protein would have a charged surface significantly different to that of the most abundant plasma proteins, such as albumin or IgG, in order to facilitate specific enrichment on a charged surface. An example of a further target analyte for which the presently disclosed method may be applied is the detection of TSH (thyroid-stimulating hormone). Accurate detection of this particular analyte could aid in the diagnosis of diseases associated with the pituitary gland; Hashimoto's disease, Graves' disease, goiter and thyroid nodules to highlight a few.

Following the ‘capture’ and ‘release’ of the target analyte via the aforementioned method, the released target analyte concentration may be determined. One method by which the target analyte concentration may be determined is via the use of an immunological assay. The various steps required in such an assay, including the formation of immuno-complexes and various wash steps are known. It is envisaged that magnetic particles may be added to the assay in order to aid the collection of the immune-complex containing the target analyte of interest to allow the determination of the concentration present.

The released target analyte concentration may be compared to a pre-determined control value. A control value is a value for the specific target analyte of interest which has previously been determined from healthy individuals (or animals). This value may constitute a range of values, determined from a pool of healthy control subjects. The test sample may then be compared to this control value to aid in the diagnosis of disease. Accurate PoC diagnosis will enable medical decisions to be made at a quicker rate than currently possible and appropriate treatment strategies devised.

The present invention also provides a fluidic device comprising a capture and release system connected to an immunodetection system via fluidic channels, structured such that the aforementioned method may be performed.

The sample containing the target analyte may undergo the advantageous pre-processing in the capture and release system of the device, producing a sample removed of interfering substances and/or a concentrated sample for inputting into the immunodetection system of the device. It is envisaged that such a system is able to increase the sensitivity of the measurement to the target analyte beyond that achievable by the immunodetection alone. The two systems comprise a fluidic system that provides low dead volume and for both devices to be built on a common fluidic platform.

To support the functionality required by the capture-and-release protocol, the fluidic housing depicted in FIG. 5 must include: a mechanism for securing the membrane in place with a fluidic seal such that fluid can be forced through the membrane instead of escaping around the side of it; multiple fluidic input and multiple fluidic output channels such that different fluids can be passed through the membrane and fluids exiting the membrane can be directed to different channels; a mechanism for valving these inlet and outlet channels; a design that minimises the dead volume associated with the device such that each new fluid that passes through the membrane is not significantly contaminated with previous fluids that have passed through it.

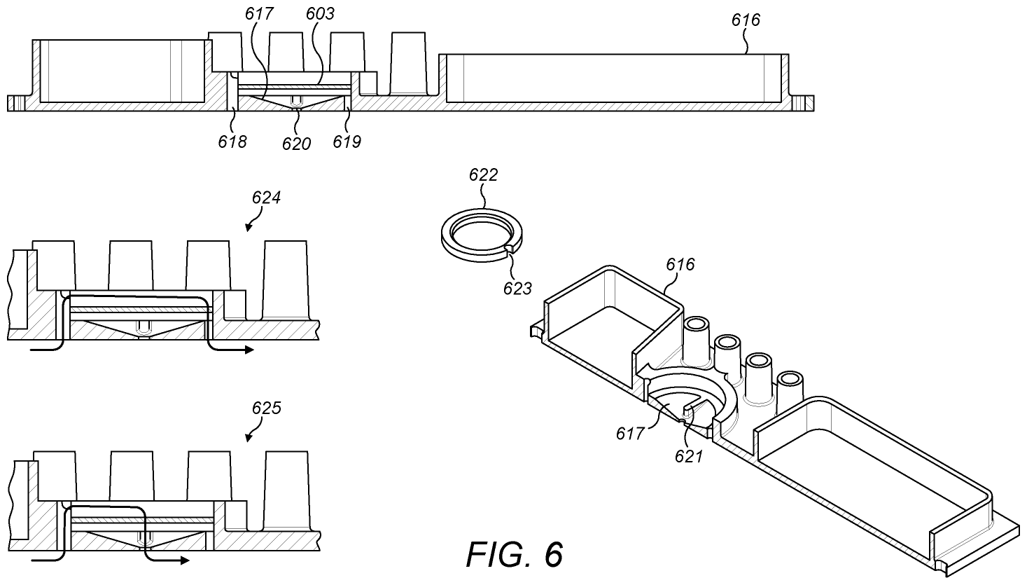

One embodiment of a fluidic device design that satisfies these requirements is shown in FIG. 6. A moulded plastic chassis 616 includes a cradle feature for the membrane to rest on 617. An input port 618 connects the lower side of the chassis to the top side of the membrane cradle. A flow through port 619 connects the top side to the cradle back to the underside of the chassis to allow fluid to flow through the upper side of the membrane without being forced through the membrane. A lower outlet port 620 connects the bottom of the membrane cradle 617 to the underside of the chassis allowing fluid that has passed through the membrane to escape 625. The membrane 203 can be sealed into the cradle and the top of this cradle sealed with a non-permeable membrane, for example a foil-backed film.

The membrane cradle 617 is formed largely of a conical section which provides a taper toward a lower outlet hole to allow for all fluid to flow out of the outlet. A series of ribs 621 are arranged to support the membrane above this outlet hole. The angle and shape of this conical section is designed so as to minimise the total volume underneath the membrane. The membrane is secured within this fluidic housing using a moulded plastic ring 622 that has an interference fit with the chassis. This can be pressed into the chassis after the desired number of membrane layers have been added to the device. The plastic ring 622 can include a notch feature 623 which aligns with the flow through port 619 when sealed into position which allows fluid to flow past the membrane without being forced through it 624. Such a function may be necessary to flush the dead volume above the membrane of one fluid before another fluid is passed through the membrane. For example, if it is desired to pass a fluid of a specific salt concentration or pH through the membrane which differs substantially from the salt or pH conditions of a previous fluid passed through the membrane, it is desired first to flush this previous fluid out of the dead volume above the membrane by using, for example, a wash fluid or air to empty this volume without forcing any further through the membrane itself.

Fluid channels can be connected to the input and output ports of this chassis on the underside by, for example, using multiple layers of elastomeric membranes. Alternatively, channels may be directly inscribed into the chassis by machining, moulding or embossing and subsequently sealed using films.

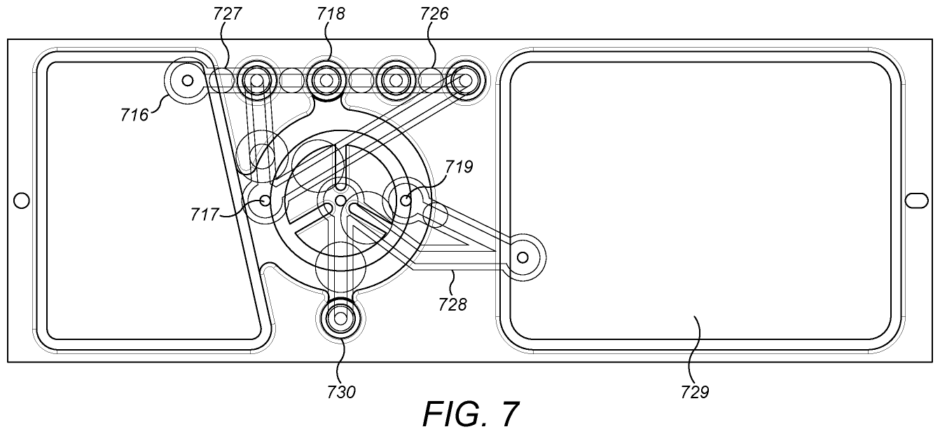

One embodiment of this fluidic device with fluidic channels is shown in FIG. 7. Multiple input ports are shown 718 through which a variety of fluids can be directed. These ports are connected by means of flexible fluidic channels to the input port of the membrane cradle 717. In particular, the arrangement of the fluid channels allows for any of one of the inputs ports to be directed to the inlet port of the membrane cradle by appropriately actuating valves 727. One of the inlet ports is connected to a fluid pot on the chassis 716 which could be used to contain one of the fluids that is desired to flow through the membrane. The flow through port 719 is connected via a fluidic channel to fluid pot on the chassis to collect the flow through 729. In this embodiment, the outlet port is connected to either the on-board fluid pot 728 or to an outlet collection port 730 depending on the valve configuration. This allows fluid that is flowed through the membrane to either pass into the waste pot or be collected into the outlet port. For example, it may be desired to direct the initial fluid from the ‘capture’ step into the on-board fluid pot 729 as well as any subsequent wash fluids. However, by then changing the valving arrangements, the elution fluid in the ‘release’ step can be collected in the outlet port.

A further embodiment of this device includes a modification of the fluidic channels to create a fluid channel with almost no dead volume and to allow the movement of fluid under both negative and positive pressure. Under positive pressure, the channel requires the positive pressure of fluid moving through the channels to open them up. If instead it is desired to pull fluid through these channels under negative pressure, for example through aspiration by a syringe, it is necessary to modify the construction of these fluid channels to prevent them sealing closed under negative pressure. As part of this invention, the second elastomeric membrane is first pre-formed along the geometry of the fluid channel which creates a ‘normally open’ fluid channel once sealed to the first membrane.

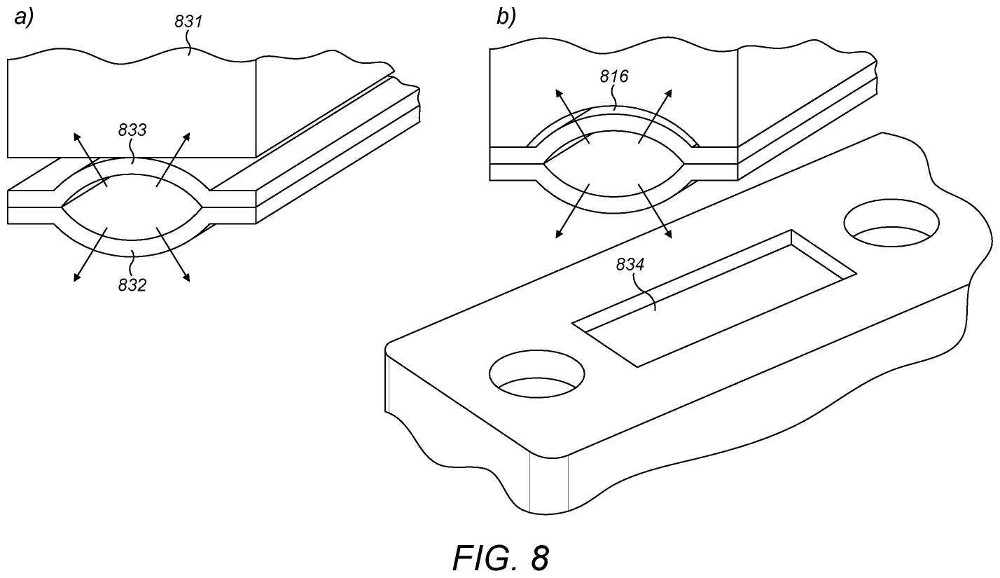

An embodiment of this modification is shown in FIG. 8. A channel in both the chassis 816 and the device holder 834 allows for the two membrane layers 832, 833 to be pre-formed to create a normally open fluid and allows for fluid to move under both positive and negative pressure. This increases the functionality of the device by allowing a syringe to move fluid through the device through aspiration as well as through positive displacement.

The immunodetection system may effectively mimic the function of an individual micro-titre well, allowing a diagnostic assay previously developed on such a format to be more easily translated onto a disposable platform.

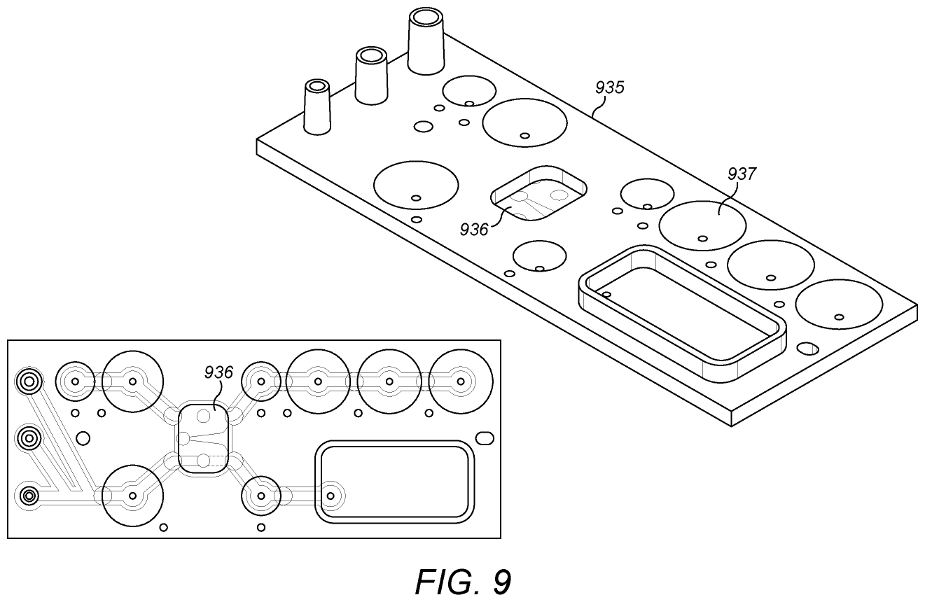

The immunodetection system may comprise the elements depicted in FIG. 9. These may include a central reaction chamber in which immune-complexes can be formed, washed and detected 936; wash fluids 937 arranged in a way in order to effect efficient washing of the central reaction chamber; an arrangement of magnets, which together with a method herein disclosed, allows for efficient collection and re-suspension of magnetic particles within the central reaction chamber 936 and a mechanism of overcoming the limited diffusion of molecules within the central reaction chamber 936 by way of enhanced agitation.

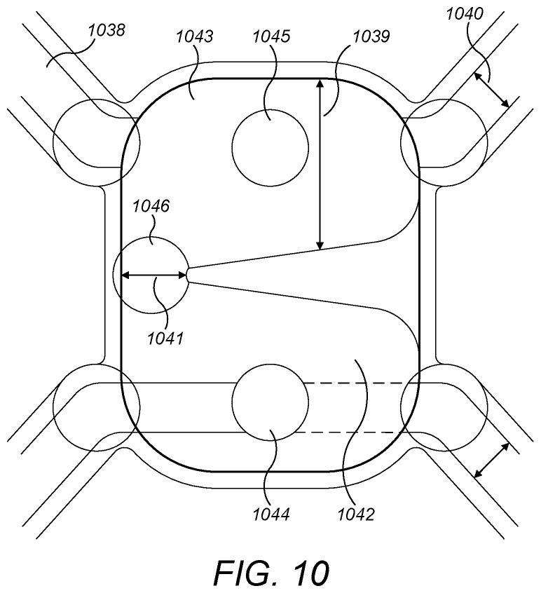

To best mimic the function of a micro-titre plate, the general structure of the fluidic device is built around a central reaction chamber 936 into which all required reaction reagents can flow into and out of when required. The device should have sufficient volume to hold all the reactants in one place; enable effective mixing of all the reactants inside the chamber; enable effective washing of unbound reagents and support pull down and re-suspension of magnetic particles. An example of an appropriate structure of the central reaction chamber is shown in FIG. 10. The chamber is formed using two layers of flexible elastomeric membrane 832, 833. A plurality of fluidic channels connects to this chamber 1038 allowing reagents to be introduced and flow through the chamber. The total volume of the chamber may be, for example, 200 uL. The present invention provides a design of a central reaction chamber 936 that can both generate effective turbulence for mixing, and in the same chamber, still permit good laminar flow to allow for an effective exchange of fluid with clean wash buffer.

The central chamber 936 should not be greater in maximum width 1039 than ˜3× the width of the channel feeding into the central chamber 1040 to preserve this laminar-like behaviour. The chamber may be elongate along the direction of fluid flow to create a rounded rectangular shape. The fluid inlet channels 1038 may be arranged into the side of the central reaction chamber 936. This is to allow the fluid flow to remain laminar as it enters the chamber. Fluid channels entering from the ‘top’ or ‘bottom’ of the chamber would experience a large increase in effective channel width, causing loss of laminar flow and poor washing.

The chamber and channel designs described above permits an effective washing protocol to be implemented on the device. Such a washing protocol may comprise the following steps: flow a volume of wash liquid through the wash input and out of the waste outlet; close the valve for the waste outlet and push in a further 100-200 uL of wash fluid into the central chamber; the ‘flow through’ wash serves to displace most of the existing fluid within the chamber out of the waste; the ‘extra wash top-up’ serves to help further dilute any remaining reagent not washed out; close all valves surrounding the central reaction chamber and use plungers to mix the liquid between the two volumes of the central reaction chamber to effectively dilute any remaining reagent hang up; repeat as necessary; following the final wash, open up the waste channel and force liquid from the central chamber out into waste to effectively evacuate the chamber of fluid.

Magnetic particles often feature in immunoassay tests and it is necessary to be able to control their movement. Within the current invention, a magnetic field can be applied to a small area of the central reaction chamber to exert a magnetic force on magnetic particles suspended within the reaction chamber, for example superparamagnetic beads suspended in a reaction liquid. This magnetic field could either be provided by, for example, an electromagnet or a permanent magnet that can be moved toward or away from the central chamber 1046. The force experienced by the magnetic particles is determined by the gradient of the magnetic field and falls approximately as the fourth power with the distance from the magnet.

One problem this creates is the very weak force far from the magnet and eventually this force is no longer strong enough to overcome the Stokes drag force on the particles exerted by the surrounding fluid which prevents them from being collected. When the reaction chamber size is more than a few millimetres in diameter, there is very little magnetic force available at the edges of the chamber to help collect the magnetic beads.

The present invention provides a means of overcoming this by use of an arrangement of magnets and motion control of plungers. Plungers arranged mechanically above the central reaction chamber 936 can be controlled to squeeze one of the lobes 1042 and 1043 making up the reaction chamber, thus forcing fluid through the neck 1041 into the other lobe. This action can help drag magnetic particles through the fluid and over a magnet positioned underneath the chamber 1046. The speed and distance with which the plunger moves to compress the lobe of the reaction chamber will determine the speed of the fluid flow and thus the drag force experienced by the suspended magnetic particles. By controlling both the speed and distance this plunger moves, by, for example, controlling the plunger via a stepper motor, the force exerted on the suspended magnetic particles can be made sufficient to move them from the edges of the chamber over the magnet but not sufficient to pull them away from the magnet once they are within a close distance of the magnet.

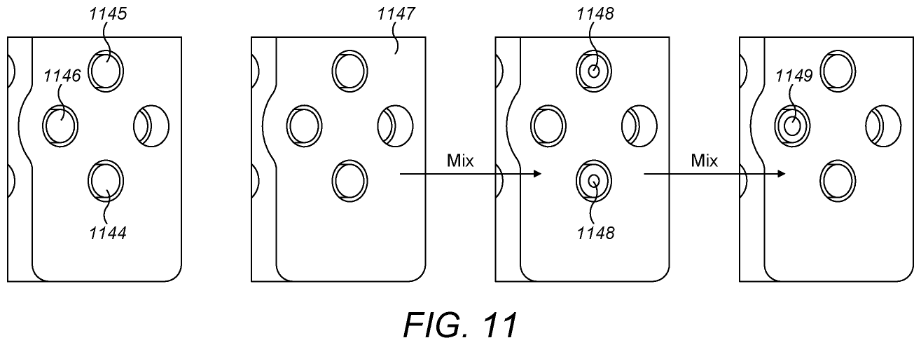

Further magnets may be added to the reaction chamber to accomplish collection of magnetic particles in stages 1044, 1045. For example, for large chambers, the force required to drag particles from the edges of the chamber may be so great that it overcomes the magnetic force available and the particles never become collected. Instead, additional magnets 1044, 1045 can be placed around the chamber. These additional magnets may be in the centre of each lobe of the chamber as well as in the neck between the two lobes 1041. These additional magnets reduce the required force from the plunger to capture magnetic particles from the edges of the chamber 1148. By then removing these magnets and then raising the centre magnet as shown in FIG. 11, all of the magnetic particles may be gathered in a single position 1149. A similar protocol could be extended for larger chambers using further magnets to collect the magnetic particles from all areas of the chamber in stages and then bring them all to one central location by selecting which magnets are raised or active and using the plungers to agitate the reaction chamber with a controlled force.

The incubation time required for the analyte to form ELISA sandwich complexes within the reaction chamber is limited by the diffusion rate of the large molecules through the reaction volume. Therefore, the use of plungers positioned above the reaction chamber provides effective mixing of reactants in the chamber.

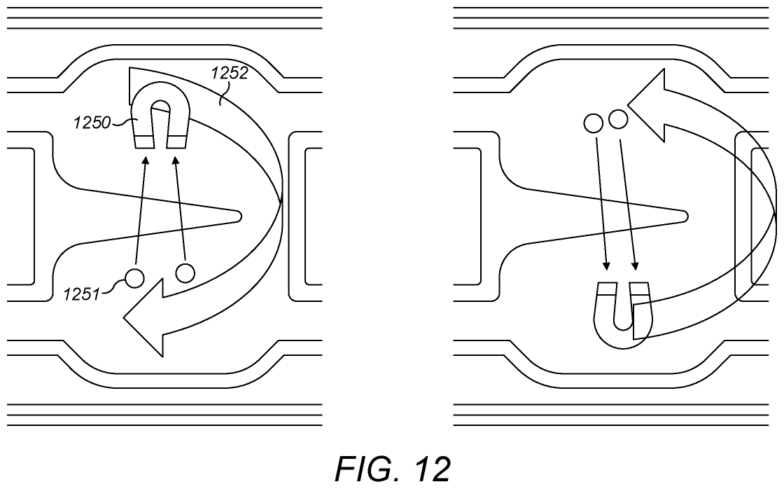

The structure of the central reaction chamber also allows for the improvement of mixing reactants containing suspended magnetic particles. An embodiment of this is shown in FIG. 12. A magnetic field placed under the upper lobe of the reaction chamber 1250 will attract magnetic particles toward it 1251. At the same time, a plunger can compress the upper lobe to force fluid toward the lower lobe 1252. This creates an opposing motion of the bulk fluid flow and the suspended magnetic particles thereby effectively exchanging the fluid immediately surrounding the magnetic particles and increasing the volume of liquid each particle is exposed to. With this method it is possible to further reduce the time required for effective mixing of all reactants in the chamber.

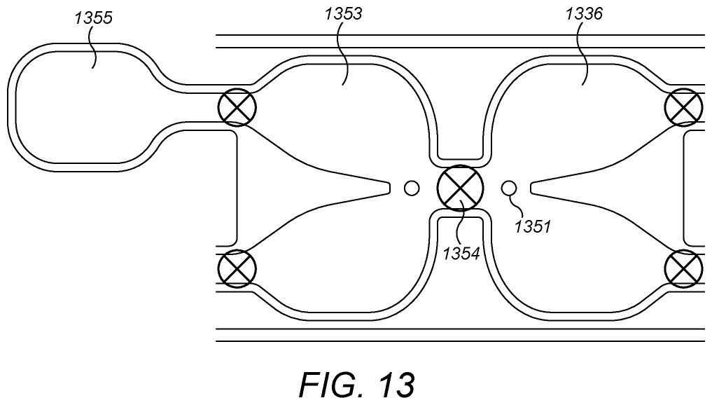

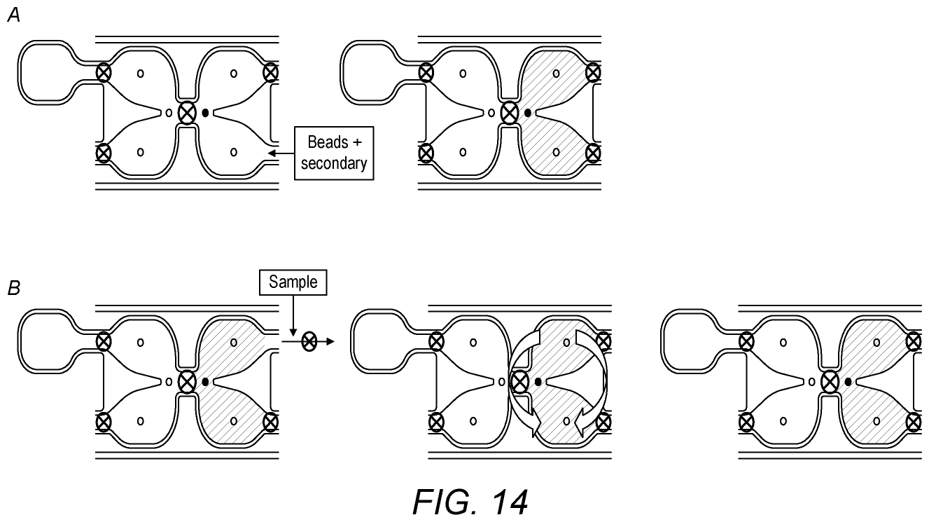

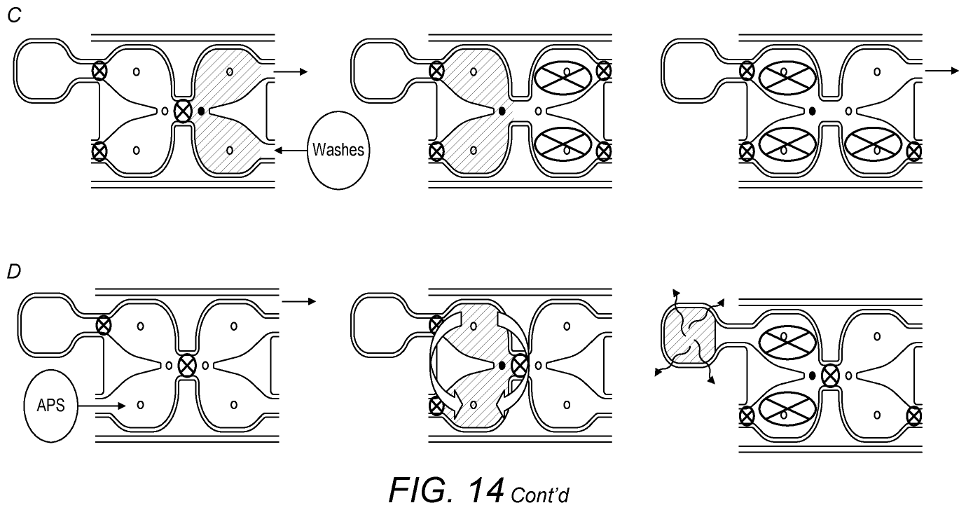

Additionally, a second chamber 1353 may be added to the device directly connected to the first chamber 1336 as shown in FIG. 13. Such an arrangement allows the contents of the reaction chamber, including any suspended magnetic particles 1351, to be transferred to this second chamber as shown in FIG. 14. This second chamber can be used to improve the signal to noise of the assay by providing a clean space, not contaminated for example by a detection enzyme.

Additionally, a third chamber 1355 may be added to the device directly connected to the second chamber 1353 described above. Such an arrangement allows for the final reactant materials to be moved to a location on the device whereby optical interrogation is easier. For example, the area directly above and below the second reaction chamber may be obstructed with mechanical actuators that make optical interrogation of the chamber difficult.

Importantly, the ionically charged surface can be co-located on the same device as the central reaction chamber. Additionally, located on the device will be storage fluid pots for the dilution buffer, wash buffer, elution buffer and waste. The device may further include syringes to enable the movement of fluid from these storage pots to where needed, e.g. to the ionically charged surface.

Different fluids can be directed to different parts of the device by selectively activating valves along the fluidic channels. Such valves can, for example, be actuated by an instrument forcing the channels closed with a press. Such a press may be pneumatically actuated, electromagnetically actuated, mechanically actuated using a stepper motor connected via a thread and lead-screw or a motor connected to a cam. The fluidic channels themselves may be made of such that the application of pressure alone is enough to close them.

The sample output from the capture and release system can be directed into the central reaction chamber of the immunodetection system. Dried reagent storage and liquid wash buffers can be stored in blisters which can, if required, be foil sealed. It is further envisaged that a second reaction chamber can be connected to the first by a fluid channel at the neck of the chamber. This second chamber can be used to receive the immune-complexes at the end of the reaction before a detection substrate is added. An example of such a substrate is a luminescent substrate, the light generated from which would be proportional to the amount of target analyte present. Possible substrates include horseradish peroxidase (HRP) and alkaline phosphatase (AP). An additional detection method that may be utilised is the detection of electrical signals generated by, for example, glucose oxidase, which is linked to the detection agent.

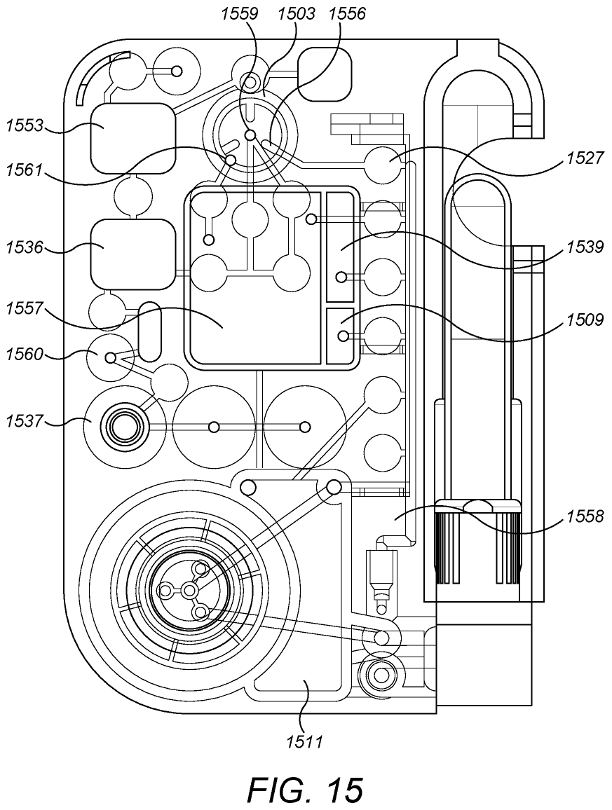

The present invention may provide a method to automate the functioning of this device, by automating the movement of fluids through the membrane. One embodiment of this method is shown in FIG. 15. A syringe that is driven by mechanical means, for example, a syringe driver or a custom stepper motor and required mechanical connection, is used to move fluid throughout the device. This syringe could, for example, be part of the disposable fluidic device 1558 and drive fluid directly by aspirating and dispensing fluid into and out of the syringe. Alternatively, the syringe could, for example, be part of the re-usable instrument in which case a fluidic connection would be made between the instrument and the consumable. Such a syringe could be either hydraulically or pneumatically coupled to the disposable device in order to move fluids around the device.

The required dilution (1511), wash (1537) and elution buffers (1509) required to operate the capture-and-release protocol as described above can be stored, for example, inside fluidic container pots on the device itself. The sample can be loaded into, for example, an empty container located on the fluidic device. By opening the correct valves (1527), the syringe can then aspirate a precisely metered sample volume into the syringe. This can then be dispensed into the fluidic pot containing the dilution buffer. By continuously aspirating and dispensing fluid between this pot and the syringe it is possible to effectively mix the sample with the dilution buffer thereby correctly adjusting the ionic conditions to enable the target analyte to be captured by the membrane. The syringe can then flow this diluted sample through the membrane into a waste collection pot thereby capturing the target analyte onto the membrane. In a similar fashion, in combination with appropriate valve configurations, the same syringe can be used to aspirate the wash and elution fluids into the syringe from their on-board pots before flowing them through the membrane. When the elution buffer is flowed through the membrane to release the target analyte, the valves can be configured to let this output into a subsequent immunoassay device or to a collection port.

As will be appreciated from the above, the present invention not only provides for a method of preparing a sample for a diagnostic/analytical assay by isolating the target analyte but also provides for a device capable of performing both the processing and the subsequent analytical assay in a format suitable for PoC testing.

1. A method of preparing a sample for carrying out a diagnostic or analytical assay, wherein a sample to be assayed is first brought into contact with a housing unit that comprises an ionically charged surface; wherein a target analyte is retained on said surface and the other constituents of the sample pass through said surface freely; releasing the target analyte from said surface by introduction of an elution fluid, which is collected via an outlet collection port for a diagnostic or analytical assay to be performed.

2. The method according to claim 1, wherein the ionically charged surface is an ion-exchange membrane or ion-exchange beads.

3. The method according to claim 2, wherein the ion-exchange membrane or the ion-exchange beads carries a positive or negative ionic charge.

4. The method according to claim 2, wherein the ion-exchange membrane or ion-exchange beads carry a weak or strong ionic exchange moiety.

5. The method according to claim 1, wherein the sample buffer comprises 10 mM buffering agent, has a pH of 7-9 and/or salt concentration of 0-50 mM, preferably wherein the buffering agent is HEPES or a phosphate-based agent.

6. The method according to claim 1, wherein the sample buffer comprises 10 mM buffering agent, has a pH of 4-6 and/or salt concentration of 0-50 mM, preferably wherein the buffering agent is MES, citrate or a phosphate-based agent.

7. The method according to claim 1, wherein the ionically charged surface retains at least 70% of the target analyte within a sample.

8. The method according to claim 1, wherein the elution fluid composition comprises 20-50 mM buffering agent, has a pH of 4-8 and a salt concentration of 250 mM-1 M, preferably wherein the buffering agent is HEPES or a phosphate-based agent.

9. The method according to claim 1, wherein the elution fluid volume is smaller than or equal to that of the sample volume.

10. The method according to claim 1, wherein the elution fluid containing the target analyte is bought into contact with the ionically charged surface multiple times.

11. The method according to claim 1, wherein the sample is a blood sample, urine sample, sweat sample, tear sample, sputum sample, faecal sample, liquid biopsy sample or spinal fluid sample.

12. The method according to claim 1, wherein the target analyte is a protein, a RNA molecule, DNA molecule, a virus, a bacterium, or part thereof.

13. The method according to claim 1, wherein the target analyte is a cardiac protein, preferably wherein the target analyte is troponin.

14. The method according to claim 1, wherein the released target analyte concentration is determined.

15. The method according to claim 1, wherein the released target analyte concentration is compared to a pre-determined control value.

16. A fluidic device comprising a capture and release system connected to an immunodetection system via fluidic channels, structured such that the method of claim 1 is performed.
